(19) United States
(12) Patent Application Publication
(10) Pub. No.: US 2023/0381353 A1
(43) Pub. Date: Nov. 30, 2023

(54) SANITIZATION SYSTEMS AND METHODS

(71) Applicant: Vahe Jarahian, Glendale, CA (US)

(72) Inventor: Vahe Jarahian, Glendale, CA (US)

(21) Appl. No.: 17/827,013

(22) Filed: May 27, 2022

(51) Int. Cl. A61L 2/10; A61L 2/24; A61L 2/18; A47L 23/02

(52) U.S. Cl. CPC A61L 2/10; A61L 2/24; A61L 2/18; A47L 23/02; A61L 2202/11; A61L 2202/14; A61L 2202/15; A61L 2202/17

(57) ABSTRACT

The disclosure features a sanitization system, sanitization kiosk, sanitization station, sanitization platform, and sanitization method for sanitizing a person's shoes. The system, kiosk, station, platform, and method can utilize one or more sanitization modalities such as cleaning, UV light sanitization, and disinfection with disinfectant solution(s) or liquid(s).

### FIELD

The present disclosure is directed to the field of sanitation. More particularly, the disclosure relates to systems and methods for sanitizing a person's shoes.

### BACKGROUND

Infection control through sanitization has become increasingly important in hospital and other clinical settings given the increase in serious nosocomial infections such as Methicillin-resistant Staphylococcus aureus (MRSA). Sanitization protocols and methods to date have not been adequate for preventing nosocomial infections or contamination in other settings, such as manufacturing or food and beverage processing.

### SUMMARY

In general, in a first aspect, the disclosure features a sanitization system. The sanitization system includes a grate designed to be stood upon by a user during use of the sanitization system, a first sanitization modality including UV light, and a second sanitization modality chosen from modalities including UV light, disinfectant solutions or liquids, and their combination. When the user stands upon the grate during use, the first sanitization modality is configured to sanitize the top and sides of the user's shoes, and the second sanitization modality is configured to sanitize the bottom of the user's shoes.

In general, in a second aspect, the disclosure features a sanitization kiosk. The sanitization kiosk includes a kiosk body, a user interface disposed at a top end of the kiosk body, a chamber disposed at a bottom end of the kiosk body, a grate covering the top of the chamber, a first sanitization modality including one or more UV lights disposed on the kiosk body above the grate, and a second sanitization modality chosen from modalities including one or more UV lights, disinfectant solutions or liquids, and their combination disposed within the chamber.

In general, in a third aspect, the disclosure features a sanitization station. The sanitization station includes a user interface, a grate disposed below the user interface, a first sanitization modality including one or more UV lights disposed above the grate, and a second sanitization modality chosen from modalities including one or more UV lights, disinfectant solutions or liquids, and their combination disposed below the grate.

In general, in a fourth aspect, the disclosure features a sanitization platform. The sanitization platform includes a chamber, one or more UV LED strips disposed within the chamber, and a grate disposed at the top of the chamber.

In general, in a fifth aspect, the disclosure features a sanitization method. The sanitization method includes sanitizing the top and sides of a pair of shoes by way of a first sanitization modality including UV light, and sanitizing the bottom of the pair of shoes by way of a second sanitization modality chosen from modalities including UV light, disinfectant solutions or liquids, and their combination.

Features of the sanitization system, sanitization kiosk, sanitization station, sanitization platform, and sanitization method can include the following. The first sanitization modality can include one or more UV lights disposed above the grate that direct UV light over the top and sides of the user's shoes. The second sanitization modality can include one or more UV lights disposed below the grate that direct UV light toward the bottom of the user's shoes. The second sanitization modality can include an absorbent medium capable of holding the disinfectant solutions or liquids disposed below the grate. The absorbent medium can be a sponge. The grate can be designed to move downward during use such that pressure from the user standing on the grate pushes the grate into the absorbent medium and releases the disinfectant solutions or liquids from the absorbent medium. A user interface can receive input for a sanitization protocol from a user including mode of sanitization, duration of sanitization, and/or intensity of sanitization for one or more stages of sanitization. A microcontroller can be in bidirectional communication with the user interface which activates the first and/or second sanitization modalities based on the input. A mat can include an adhesive material capable of removing particular matter from the bottom of the user's shoes when stood upon. The one or more UV lights of the first sanitization modality and second sanitization modality can include UV LEDs. A reservoir can be disposed above the chamber and can be designed to hold and dispense the disinfectant solutions or liquids into the chamber. One or more pressure or motion sensors can be configured to sense a user's pressure or motion during use of the sanitization system, station, platform, or kiosk. The sensors with a timer can determine a duration of use of the sanitization system, station, platform, or kiosk. A door lock/unlock activator can be configured to unlock or lock a door. The microcontroller can receive input from the one or more pressure or motion sensors and the timer and send an output to the door lock/unlock activator to unlock the door when a threshold duration of use of the sanitization system, station, platform, or kiosk is determined. A door lock/unlock indicator can display a locked or unlocked status of the door. Particulate matter can be removed from the bottom of the user's shoes by way of a mat including an adhesive. A sanitization protocol including a first stage sanitization modality, a second stage sanitization modality, and/or a third stage sanitization modality, can be chosen and activated, the modalities chosen from cleaning, UV light sanitization, and disinfection with disinfectant solution(s) or liquid(s) and combinations thereof.

It should be understood that the sanitization system, kiosk, station, platform and method are not to be considered limitations on the invention defined by the claims. The featured sanitization system, kiosk, station, platform, and method can be implemented in one or more ways using one or more features depicted in the drawings, described in the detailed description, and set forth in the claims.

### BRIEF DESCRIPTION OF THE DRAWINGS

The accompanying drawings illustrate certain aspects and principles of the implementations set forth, and should not be construed as limiting.

FIG. 1 is a schematic diagram of a control system for a sanitization system according to one implementation.

FIGS. 2A and 2B are diagrams of a sanitization kiosk according to one implementation.

FIG. 3 is a diagram of grate, chamber, and UV LED arrays according to one implementation, that can be employed together in a sanitization kiosk or sanitization station or can be used as a stand-alone sanitization system as a sanitization platform.

FIGS. 4A and 4B are diagrams of a sanitization station according to one implementation.

### DETAILED DESCRIPTION

Reference will now be made in detail to various illustrative implementations. It is to be understood that the following discussion of the implementations is not intended to be limiting.

“Sanitization” and “sanitizing” used in the context of systems, kiosks, stations, platforms, or methods described herein are used interchangeably to mean systems, kiosks, stations, platforms, or methods that reduce pathogenic agents or other microorganisms to acceptable levels or eliminate them, such as reducing or eliminating them on shoe surfaces. What the acceptable level is depends on the health requirements of the environment where sanitization occurs.

The disclosure provides specialized sanitization systems capable of sanitizing a user's shoes as they enter a location. The sanitization system includes multiple sanitizing features or modalities capable of sanitizing the surfaces of the user's shoes, such as the top, bottom, back, and sides. The features or modalities can have one more sanitizing activities including germicidal activity, sporicidal activity, virucidal activity, moldicidal activity, fungicidal activity, mildewcidal activity, protozocidal activity, yeasticidal activity, and algaecidal activity, or combinations thereof. The features or modalities can have one or more cleaning activities, or one or more disinfecting activities, or one or more sterilizing activities. The features or modalities can reduce pathogens such as bacteria, spores, viruses, mold, fungi, mildew, protozoa, yeast, algae, and/or other pathogens to acceptable levels on the surfaces of the user's shoes. Implementations can provide a 90% reduction in pathogens from shoe surfaces, 99% reduction from shoe surfaces, 99.9% reduction from shoe surfaces, 99.99% reduction from shoe surfaces, 99.999% reduction from shoe surfaces, or a 99.9999% reduction from shoe surfaces, up to the point where the pathogens are virtually eliminated from the shoe surfaces, based upon what level of sanitization is deemed acceptable. UV Doses (mJ/cm², or J/m²) for various reduction levels of different types and species of microorganisms can be found in references or summaries published on the Internet, such as the websites of the manufacturers of UV equipment including American Air and Water® (Hilton Head Island, South Carolina, U.S.A.), ClorDiSys Solutions Inc. (Lebanon, New Jersey, U.S.A.), and UV Light Technology Limited (Birmingham, U.K.). In general, these doses range from about 1 to about 500 mJ/cm². Implementations of the sanitization system include mats with adhesives that remove particulate material, sanitizing UV radiation, and disinfecting antiviral and/or antibacterial solutions or liquids that can be applied to user's shoes when the user enters the sanitization system. The sanitization system can be or include a mat, grate, platform, sanitizing kiosk, sanitizing station, or combination thereof, that incorporates one or more of the sanitizing features or modalities. The UV radiation can be implemented in the form of UV LEDs, UV fluorescent light bulbs, or UV lamps that can emit light in the UV-C portion of the electromagnetic spectrum (i.e., 100-280 nm wavelengths) which is known to have the highest sanitizing activity. The disinfecting antiviral and/or antibacterial solutions or liquids can include one or more disinfectant compounds including alcohols (e.g., ethyl alcohol, isopropyl alcohol), chlorine compounds (e.g., sodium hypochlorite (active ingredient of bleach)), quaternary ammonium compounds (e.g., benzalkonium chloride), phenolics (e.g., carbolic acid, o-phenylphenol, chloroxylenol (active ingredient of DETTOL™) iodophors (e.g., povidone-iodine (active ingredient of BETADINE®)), and hydrogen peroxide. The concentrations of the active ingredients required to achieve sanitization, and discussion of other active ingredients that can be included can be found in references available to the skilled artisan, such as Russell, Hugo & Ayliffe's Principles and Practice of Disinfection, Preservation and Sterilization, 5th ed. (ed. A. P. Fraise, J. Maillard, S. A. Satter), John Wiley & Sons, New York, incorporated by reference in its entirety.

One implementation of the sanitization system includes a mat that can be stepped on by a user at an initial stage of sanitization to provide cleaning. The material of the mat can include an adhesive that removes particulate matter from the undersides of the user's shoes, prior to the user entering the next stages of a sanitizing protocol. Another implementation provides a grate disposed over a chamber that houses one or more sanitizing modality. The grate is designed and dimensioned to support the weight of a person standing on the grate. The sanitizing modality within the chamber underneath the grate can include UV-emitting lights. The UV-emitting lights can be implemented as an array or arrays of UV LEDs such as light strips, one or more UV fluorescent light bulbs, or one or more UV lamps that direct sanitizing UV radiation on the underside of a user's shoes when the user steps on the grate. The sanitizing modality within the chamber underneath the grate can include a sponge or other absorbent medium capable of holding disinfectant solutions or liquids. The disinfectant solutions or liquids can be stored in a reservoir above the chamber and can replenish the absorbent medium through a tube fed through gravity by way of a controlled valve, or through a pump that moves the disinfectant solutions or liquids into the chamber. The valve and/or pump can be controlled manually or through a motor or actuator. The grate is designed to move downward into the chamber when a user stands on the grate, such that the user's weight pushes the grate into the sponge or other absorbent medium thereby compressing the absorbent medium and releasing the disinfectant solutions or liquids to flow above the grate through the spaces provided by the grate and coat the undersides of the user's shoes. Alternatively, the grate can be stationary and rollers made with an absorbent medium can press upward to release the disinfectant directly underneath the grate. Alternatively, the disinfectant solutions or liquids can be sprayed up through the grate to contact the user's shoe bottoms when the user stands on the grate. Standing on the grate can activate a pressure sensor which triggers the rollers or activates a pump to spray the disinfectant solutions or liquids upward through the grate. The sanitizing modality can include a combination of the UV-emitting lights and disinfectant solutions or liquids. The undersides of the user's shoes are sanitized by the combination of UV radiation and disinfectant solutions/liquids when the user stands on the grate. Another implementation of the sanitization system includes UV-emitting lights that direct sanitizing UV radiation above the grate toward the top and sides of the user's shoes. The UV-emitting lights can be designed to move at different angles to direct sanitizing UV radiation at different portions of the tops and sides of the user's shoes (e.g., a fixture holding the UV-emitting lights can move or detach, and the user can direct the light at different angles over the shoe surface manually) or can be provided in a hemispherical dome-like structure that can be lowered over the user's shoes which allows the UV radiation to be directed to the sides as well as the top of the shoes. The UV-emitting lights can be provided as an array having an appropriate number, density, and configuration for providing sufficient energy for sanitizing the shoe surfaces under the array, depending on the UV-emitting light source used. As such, the sanitization system allows for the relatively complete sanitization of the surfaces of the user's shoes (underside, top, and sides and back). Another implementation of the sanitization system includes a sanitizing kiosk or sanitizing station that incorporates one or more of the above features and a user interface that allows a user to design a sanitization protocol by choosing which sanitizing features he or she wants to be activated and in what order. The user interface can allow for one or more programmed sanitization treatments where a user would be able to select a desired sanitization range including a duration of sanitization and/or intensity of sanitization. For example, if the user is first entering a facility from the outside world, the user would select the highest levels of sanitization duration and/or intensity. On the other side of the spectrum, if a user is returning to a room he or she previously exited shortly before, and never left the building, then the user could select a less rigorous sanitization treatment duration and/or intensity. Alternatively, or in addition, the user interface can calculate duration and intensity of each mode of sanitation based on input of a desired sanitation level (e.g., 90% reduction, 99% reduction, 99.9% reduction, 99.99% reduction, 99.999% reduction, 99.9999% reduction) for a particular microorganism. The levels of sanitization can be determined by administering sanitizing UV radiation and/or disinfectant solutions or liquids at various dosages over various time periods (e.g., 30 seconds, 1 minute, 2 minutes, 3 minutes, 4 minutes, 5 minutes, 10 minutes) that the user engages with the sanitizing system. The sanitizing system can further include a mechanism that locks a door to another room or location prior to completion of sanitization and unlocks the door after sanitization. The entry door can unlock only when a user has spent a requisite period of time on the sanitizing mat, grate, platform, kiosk and/or station for full sanitization to occur. Another implementation of the sanitization system includes a motion detector which activates one or more of the sanitizing features when motion is detected. Alternatively, or in addition, the sanitizing features can be activated by a pressure-activated switch once a user steps on the mat and/or grate. The motion detector and/or pressure detector can also determine with a timer how long the user is standing upon the mat, grate, platform, kiosk, and/or station to determine whether a sufficient duration of sanitization has occurred. An additional feature can include a signaling device to signal to a user through audio or visual output that they need to remain in place upon the mat or grate while the sanitization system is working. The sanitization system can apply sanitization in a sanitization protocol at different stages, such that a first stage would be one form of sanitization (e.g., a cleaning mechanism such as an adhesive that removes particulate matter from the underside of the shoe), followed by a second stage of sanitization (e.g., UV light), followed by a third stage of sanitization (e.g., disinfectant solution or liquid). The sanitizing system can include a control system which includes a microcontroller or processor for controlling various functions of the sanitizing system. Also provided are sanitization methods that include sanitizing the top and sides of a pair of shoes by way of a first sanitization modality including UV light, and sanitizing the bottom of the pair of shoes by way of a second sanitization modality chosen from modalities including UV light, disinfectant solutions or liquids, and their combination. The methods can further include first removing particulate matter from the bottom of a pair of shoes by way of a mat including an adhesive. The methods can further include choosing and activating a sanitization protocol including a first stage sanitization modality, a second stage sanitization modality, and/or a third stage sanitization modality, the modalities chosen from cleaning, UV light sanitization, and disinfection with disinfectant solution(s) or liquid(s), or combinations thereof. The sanitization systems, methods, and their features can be used in settings where infection and contamination control are critical, such as hospitals and manufacturing. These and other implementations will be depicted in the following figures.

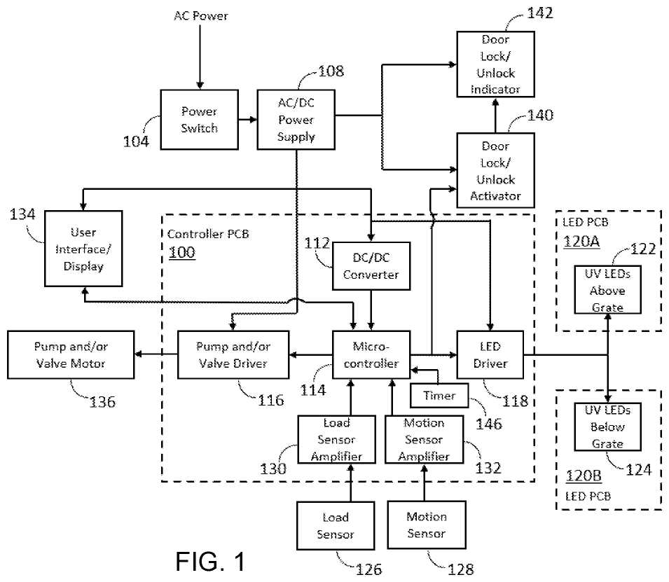

FIG. 1 shows a control system for a sanitizing system (e.g., kiosk, platform, station) according to one implementation. The control system can include a controller printed circuit board (PCB) 100 and LED PCBs 120A and 120B. A microcontroller 114 (i.e., processor) is at the center of the control system and can control various aspects of the functions of the sanitizing system. The microcontroller 114 controls an LED Driver 118 which controls one or more UV LEDs 122 disposed above the grate or UV LEDs 124 disposed below the grate. The microcontroller 114 also controls a driver 116 which controls a pump and/or valve motor 136 for dispensing disinfectant solutions or liquids. The microcontroller 114 also controls a door lock/unlock activator 140 that switches the lock on a door between a locked and unlocked position. The door lock/unlock activator 140 controls a door lock/unlock indicator 142 which can be a visual indicator such as a light or illuminated sign which signals the lock/unlock status of the door. The microcontroller 114 also receives input from a load sensor 126 and motion sensor 128 which are amplified by a load sensor amplifier 130 and motion sensor amplifier 132 prior to being received by the microcontroller 114. The microcontroller also further receives input from a timer 146. A user interface/display 134 is in bidirectional communication with the microcontroller 114. A power switch 104 controls an AC/DC power supply 108 which sends power to various components including the user interface/display 134, pump and/or valve driver 116, LED driver 118, door lock/unlock activator 140, and door lock/unlock indicator 142. Power from the power supply 108 passes through DC/DC converter 112 prior to supplying microcontroller 114.

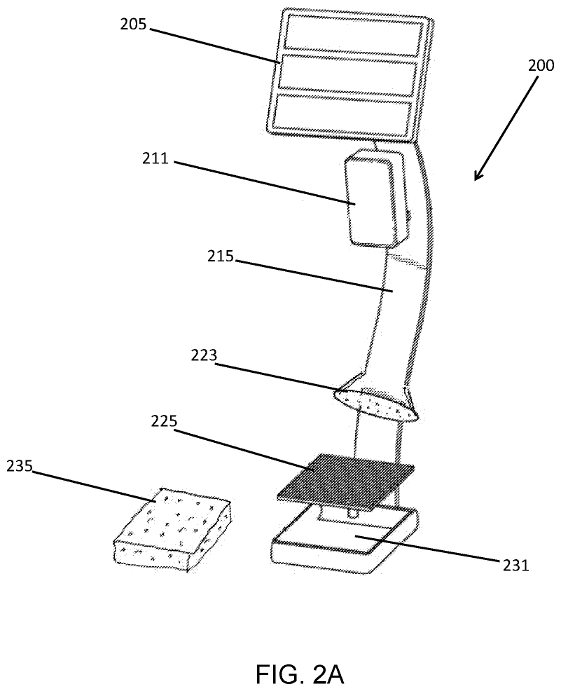

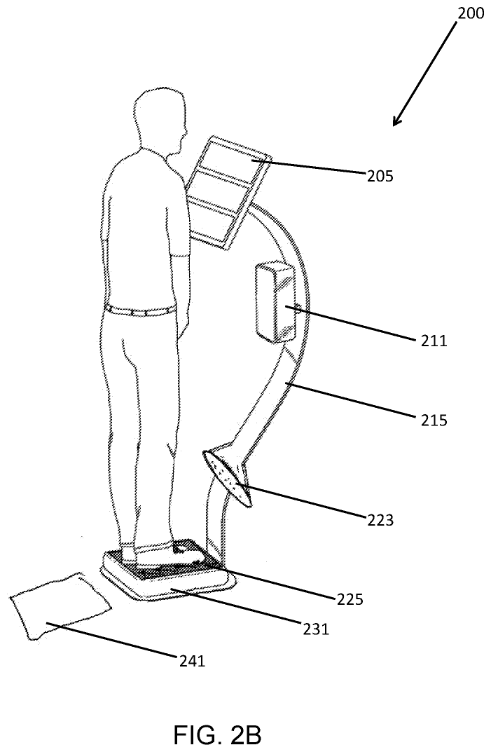

FIGS. 2A and 2B show a sanitizing system implemented as a kiosk 200. The sanitizing kiosk includes a kiosk body 215, such as an elongated curved structure as shown (although other dimensions of the kiosk body are contemplated), made of a strong thermoplastic material (although other materials such as metal or metal alloy are contemplated). A user interface/display 205 is disposed at the top end of the kiosk body 215, and a base chamber 231 sits at the bottom of the kiosk body 215. Below the user interface 205 is a reservoir 211 that holds disinfectant solutions or liquids. The disinfectant solutions or liquids can pass from the reservoir through a tube (not shown) that passes through the body 215 of the sanitizing kiosk 200 to the base chamber 231 of the kiosk. Dispensing of the disinfectant solutions or liquids can be controlled by a valve and/or pump (not shown) behind the reservoir 211. The valve and/or pump can be controlled manually by the user or through a motor. The sanitizing kiosk also includes a grate 225 for a user to stand on that sits above and covers the base chamber 231. Above the grate is an array of UV LEDs 223 for sanitizing the top and sides of the user's shoes. The array of UV LEDs 223 can be mounted on a separate structure and moveable or detachable to direct UV light to the top and sides of the user's shoes at various angles and can be activated to turn on through a pressure and/or motion sensor with timer (not shown). Alternatively, the array of UV LEDs can be provided in a dome-like structure that can be lowered over the user's shoes to provide such side coverage. The array of UV LEDs can have an appropriate number, density, and configuration of UV LEDs (e.g., 10×8, 15×20, 20×40, 50×100) for providing sufficient energy for sanitizing the shoe surfaces under the array. The grate 225 is designed to lower into the base chamber 231 of the sanitizing kiosk 200 when receiving the weight of a user standing on the grate 225. The grate 225 can be supported underneath by springs in the base chamber 231 which compress under the weight of the user to the point of full compression of the springs. A sponge or other absorbent medium 235 (shown outside the chamber to facilitate visualization) sits in the chamber 231 and holds the disinfectant solutions or liquids. When the user stands on the grate, the grate compresses the sponge thus releasing disinfectant solutions or liquids which flow through the openings or spaces in the grate and come in contact with the bottom of the user's shoes. The chamber 231 can have an inner wall designed to prevent full compression of the sponge, allowing only partial compression (e.g., 0.5-inch, 1-inch, 1.5-inch, 2-inch) to release the disinfectant. Other implementations for applying the disinfectant are contemplated, including rollers made with an absorbent medium which press upward into the grate and release the disinfectant, or spraying devices which spray the disinfectant upward through the grate in the area where the user is standing. The UV LEDs 223 and/or disinfectant can be activated to turn on through a pressure and/or motion sensor with timer (not shown) which ensures the user is standing on the grate for a sufficient amount of time. A control system such as the system shown in FIG. 1, or portions thereof, can be hidden within or behind the kiosk body. FIG. 2B shows the sanitizing kiosk 200 during use with a user standing on the grate 225. FIG. 2B also shows a tacky mat 241 with an adhesive that the user can step on prior to stepping on the grate as an initial cleaning stage of sanitization. The adhesive of the tacky mat 241 can pull of dirt, debris, and other particulate matter from the bottom of the user's shoes prior to the user stepping on the grate 225.

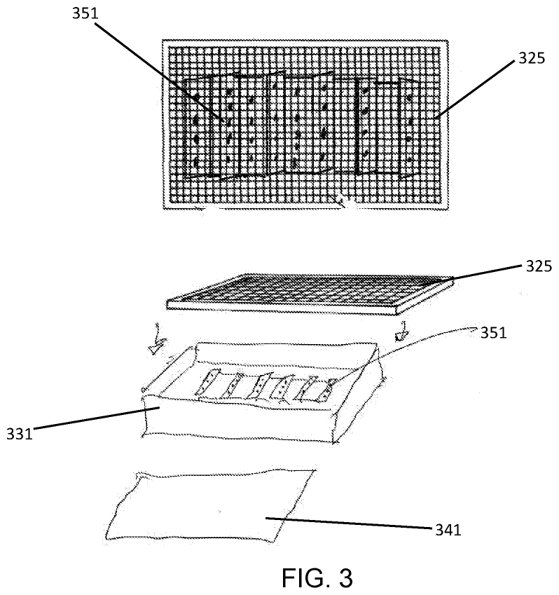

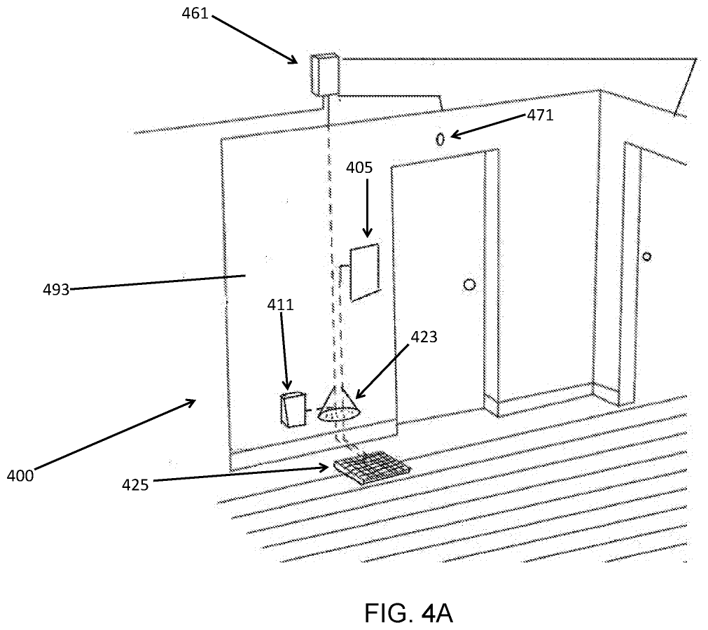

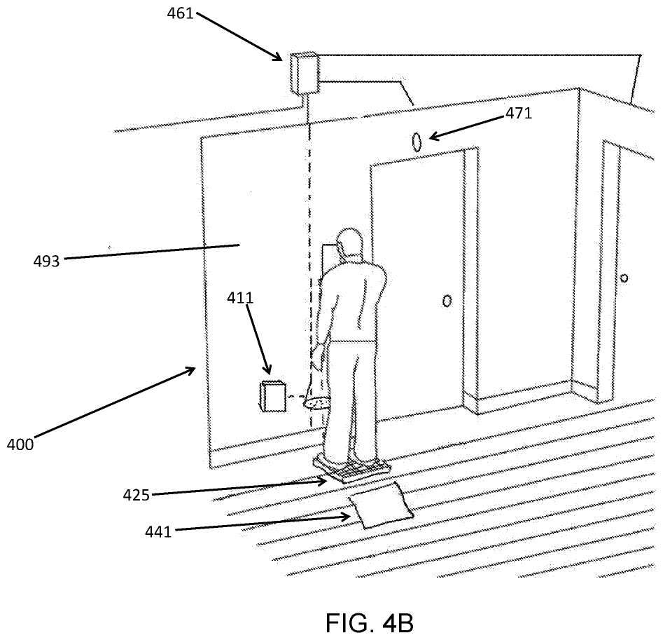

FIG. 3 shows another implementation of a grate 325 of a sanitizing system. In this implementation, arrays of UV LEDs 351 are disposed in the base chamber 331 as UV LED light strips. The grate 325 covers the base chamber 331 during use and the UV LEDs 351 sanitize the bottom of the user's shoes when the user stands on the grate 325 by directing light through the spaces in the grate. In an alternative embodiment, the UV LEDs 351 can also be mounted directly to the grate 325 rather than in the chamber 331. The UV LEDs 351 can be activated to turn on through a pressure and/or motion sensor with timer (not shown) that determines when and how long the user is standing on the grate 325. In some implementations, the base chamber 331 holds a combination of UV LEDs 351 and a sponge or other absorbent medium holding disinfectant solutions or liquids. In some implementations, the base chamber 331 holds the absorbent medium holding disinfectant solutions or liquids. The grate 325, base chamber 331, and UV LEDs 351 can be implemented in the sanitizing kiosk 200 of FIGS. 2A and 2B or the sanitizing station 400 of FIGS. 4A and 4B, described below. Alternatively, or in addition, the grate 325, base chamber 331, and UV LEDs 351 can be employed as a stand-alone system as a sanitization platform for disinfecting the bottom of a user's shoes. The grate 325 can be dimensioned to accommodate a user's shoes standing on the grate and can be made of a metal or metal alloy with appropriate spacing to allow UV light to penetrate. In some implementations, a strong, transparent plastic material or glass that allows UV light to freely penetrate is used instead of the grate.

FIGS. 4A and 4B show another implementation of a sanitizing system as a station 400 with one or more components mounted on a wall of a structure. In this implementation, components of the sanitization station 400, including the user interface/display 405, reservoir 211 for holding disinfectant solutions or liquids below the user interface, and array of UV LEDs 423 for sanitizing the top and sides of the user's shoes are attached to a wall 493, with various wires supplying the different components and conduits (e.g., tube for transporting disinfectant) of the system 400 hidden within or behind the wall 493. The grate 425 sits on the floor beneath the other components mounted on the wall 493. The grate 425 of the sanitizing station 400 can sit above a chamber having an array of UV LEDs as light strips, absorbent medium with disinfectant solutions or liquids, or some combination of these for sanitizing the bottom of the user's shoes. Also shown are a door lock/unlock activator 461 which is controlled by a combination of pressure and/or motion sensors and a timer for ensuring the user spends an adequate amount of time on the grate 425 of the sanitizing system prior to unlocking the door, and a door lock/unlock indicator 471 which displays the locked/unlocked status of the door. A control system such as the system shown in FIG. 1, or portions thereof, can be hidden within or behind the wall. The sanitizing station 400 is also shown during use in FIG. 4B with a user standing on the grate 425, after standing on the tacky mat 441 to clean the underside of the user's shoes.

The user interface of the sanitizing kiosk 200 or sanitizing station 400 can be implemented as a touchscreen which displays specific icons, fields, and other features which are selectable by a user of the sanitizing system or implemented as a traditional computer screen where the features are selectable by way of a mouse or other input device and provide for input. Selection of the icons and other features by a user activates certain functions of the sanitizing system. The functions of the sanitizing system can be executable as computer program instructions stored on one or more computer-readable memory, or can be implemented as firmware or hardware (e.g., circuitry) or some combination of these. These computer program instructions may be provided to one or more processor(s) of the sanitizing system such as a microcontroller such that the instructions, which execute via the processor or processors create means for implementing the functions of the sanitizing system. As such, functions of the sanitizing system described herein can be controlled through computer-readable code, computer-readable instructions, computer-executable instructions, or “software” performed by one or more processor(s). Such software can be loaded onto a memory or memories of the sanitizing system as an application or program (or multiple applications and/or programs) capable of executing one or more functions of the sanitizing system. The computer-readable code, computer-readable instructions, computer-executable instructions, or “software” can be organized into routines, subroutines, procedures, objects, methods, functions, or any other organization of computer-executable instructions that is known or becomes known to a skilled artisan in light of this disclosure, and can be programmed in any suitable programming language, including PHP, HTML, XML, XHTML, JavaScript, C, C#, C++, Java, Python, Perl, Ruby, Swift, Visual Basic, and Objective C. By such programming, the computer-readable code, computer-readable instructions, computer-executable instructions, or “software” instruct one or more processors of the sanitizing system to carry out the functions of the sanitizing system. Input from the user interface can be stored in the sanitizing system's memory, and can include selection of various modalities of the sanitizing system including UV sanitization, the use of disinfectant solutions or liquids, or their combination at different stages (first UV then disinfectant solution; first disinfectant solution then UV; or simultaneous), a duration of sanitization (e.g., 30 seconds, 1 minute, 2 minutes, 5 minutes, 10 minutes), an intensity of sanitization, and/or a desired sanitation level. The intensity of sanitization can be a controllable level of output of UV energy measured as a dose in terms of Joules per square meter or millijoules per square centimeter. Alternatively, the intensity of sanitization can be controlled by adjusting the strength of the disinfectant solutions or liquids by controlling an amount of water or other solvent used to dilute the active ingredient(s) of the disinfectant solutions or liquids. The water or other solvent can be stored in a separate reservoir. The input can be entered into the sanitizing system through one or more data entry fields, check boxes, lists, or pull-downs displayed on the user interface which allow the user to input information related to the modes of operation, duration of sanitization, intensity of sanitization, and/or desired sanitation level of the sanitizing system. The input can direct a processor (e.g., the microcontroller 114 of FIG. 1) to turn on and off certain functions, such as turning on or off UV LEDs 122 and 124 or activating a pump and/or valve 136 or multiple pumps or valves for dispensing disinfectant solutions or liquids at various strengths. The user interface can display messages indicating that sanitization is in progress, the mode of sanitization being used, a timer indicating how much sanitization has progressed, a message instructing the user to remain standing on the grate and/or a message indicating sanitization has been completed. The memory of the sanitization system can be implemented through non-transitory computer-readable storage media such as RAM. As used in the context of this specification, a “non-transitory computer-readable storage medium (or media)” may include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM.

Alternatives to the user interface include a computer or portable computing device such as a desktop computer, laptop computer, tablet, gaming device, PDA or smartphone. The user interface functions can be provided as an application from cloud storage services providing retail application downloading to the user's computer or portable computing device through a network connection for hosting on the user's computer or computing device. The network can use any suitable network protocol, including IP, UDP, or ICMP, and may be any suitable wired or wireless network including any local area network, wide area network, Internet network, intranet network, telecommunications network, Wi-Fi enabled network, or BLUETOOTH® enabled network. Examples of suitable Internet-accessible sources include the Apple Store, Google Play, and other sites that make software applications and other downloads available for purchase or license. As such, instead of a specialized display, the user interface functions described above can be performed on the user's own computer or portable computing device through a downloaded application. The computer or computing device can be connected to other components of the sanitization system such as the microcontroller and direct their control and function by way of a wired connection (universal serial bus, or USB) or a wireless connection (Wi-Fi, BLUETOOTH®). The computers, computing devices, or systems including the computers or computing devices can include a variety of components known in the art, including one or more processor(s), a volatile memory, a non-volatile memory, standard I/O interfaces such as one or more interfaces including a universal serial bus (USB) port, an HDMI or HDMI ARC port, an optical port, an ethernet port, and/or a serial port, a hard drive, a disk drive, a CD-ROM drive, a motherboard, a printed circuit board (PCB), circuitry for enabling a wireless connection or communication to another device, such as a BLUETOOTH® board, a Wi-Fi board, or a transmitter-receiver for mobile telecommunications, a data bus, an address bus, a control bus, and/or one or more user interface devices including a display, keyboard, keypad, trackpad, mouse, control panel, touchscreen display, speaker, camera, and/or microphone. Portable computers/computing devices can include a GPS receiver. The computers, computing devices, or systems thereof can be equipped with an operating system implemented as software or firmware. As can be appreciated, the computers, computing devices, or systems may differ in their inclusion, exclusion, or configuration of components according to their individual applications or designs.

The present disclosure has described particular implementations having various features. In light of the disclosure provided above, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. Further, one skilled in the art will recognize that one or more features described for one implementation (such as a sanitization kiosk) can be used interchangeably with features described for another implementation (such as a sanitization station or method). When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A sanitization system comprising:
a grate designed to be stood upon by a user during use of the sanitization system;
a first sanitization modality comprising UV light; and
a second sanitization modality chosen from modalities comprising UV light, disinfectant solutions or liquids, and their combination;
wherein when the user stands upon the grate during use:
the first sanitization modality is configured to sanitize the top and sides of the user's shoes; and
the second sanitization modality is configured to sanitize the bottom of the user's shoes.

2. The sanitization system of claim 1, wherein the first sanitization modality comprises one or more UV lights disposed above the grate that direct UV light over the top and sides of the user's shoes.

3. The sanitization system of claim 1, wherein the second sanitization modality comprises one or more UV lights disposed below the grate that direct UV light toward the bottom of the user's shoes.

4. The sanitization system of claim 1, wherein the second sanitization modality comprises an absorbent medium capable of holding the disinfectant solutions or liquids disposed below the grate.

5. The sanitization system of claim 4, wherein the grate is designed to move downward during use such that pressure from the user standing on the grate pushes the grate into the absorbent medium and releases the disinfectant solutions or liquids from the absorbent medium.

6. The sanitization system of claim 1, further comprising a user interface which receives input for a sanitization protocol from a user comprising mode of sanitization, duration of sanitization, and/or intensity of sanitization for one or more stages of sanitization.

7. The sanitization system of claim 6, further comprising a microcontroller in bidirectional communication with the user interface which activates the first and/or second sanitization modalities based on the input.

8. The sanitization system of claim 1, further comprising a mat comprising an adhesive material capable of removing particular matter from the bottom of the user's shoes when stood upon.

9. A sanitization kiosk comprising:
a kiosk body;
a user interface disposed at a top end of the kiosk body;
a chamber disposed at a bottom end of the kiosk body;
a grate covering the top of the chamber;
a first sanitization modality comprising one or more UV lights disposed on the kiosk body above the grate; and
a second sanitization modality chosen from modalities comprising one or more UV lights, disinfectant solutions or liquids, and their combination disposed within the chamber.

10. The sanitization kiosk of claim 9, wherein the one or more UV lights of the first sanitization modality and second sanitization modality comprise UV LEDs.

11. The sanitization kiosk of claim 9, wherein the disinfectant solutions or liquids are held in an absorbent medium.

12. The sanitization kiosk of claim 11, wherein the absorbent medium is a sponge.

13. The sanitization kiosk of claim 11, wherein the grate is designed to move downward during use such that pressure from the user standing on the grate pushes the grate into the absorbent medium and releases the disinfectant solutions or liquids from the absorbent medium.

14. The sanitization kiosk of claim 9, further comprising a reservoir disposed on the kiosk body above the chamber, the reservoir designed to hold and dispense the disinfectant solutions or liquids into the ch

15. A sanitization platform comprising:
a chamber;
one or more UV LED strips disposed within the chamber; and
a grate disposed at the top of the chamber.
